(19) United States
(12) Patent Application Publication
(10) Pub. No.: US 2018/0110792 A1
(43) Pub. Date: Apr. 26, 2018

(54) COMPOSITIONS AND METHODS FOR INHIBITING GLYCATION REACTIONS

(71) Applicant: Access Business Group International LLC, Ada, MI (US)

(72) Inventors: Michael Chop, Los Angeles, CA (US); Dawna Venzon, Seal Beach, CA (US); Katie Throop, Mission Viejo, CA (US); Kelly Glynn, Grand Rapids, MI (US); John Rebhun, Greenville, MI (US)

(21) Appl. No.: 15/791,939

(22) Filed: Oct. 24, 2017

Related U.S. Application Data
(60) Provisional application No. 62/412,449, filed on Oct. 25, 2016.

(51) Int. Cl. A61K 31/7004; A61K 36/28; A61K 36/185; A23L 33/105; A23L 33/125

(52) U.S. Cl. CPC A61K 31/7004; A61K 36/28; A23V 2002/00; A23L 33/105; A23L 33/125; A61K 36/185

(57) ABSTRACT

Dietary supplement compositions including L-arabinose and extracts of chamomile and blackcurrant are described. Methods of using the same for managing blood sugar levels and/or decreasing glycation reaction in a body and skin of a subject are also described.

### RELATED APPLICATIONS

The present patent document claims the benefit of the filing date under 35 U.S.C. § 119(e) of Provisional U.S. Patent Application Ser. No. 62/412,449, filed Oct. 25, 2016, which is hereby incorporated by reference.

### BACKGROUND

### 1. Technical Field

The disclosure relates generally to dietary supplements comprising L-arabinose and one or more select compounds or extracts. The disclosure relates to a dietary supplement for reducing blood sugar and inhibiting glycation reaction(s) in the body and skin that result in Advanced Glycation End Products (AGEs). The composition of the disclosure combines a nutritionally effective amount of L-arabinose, and extracts of chamomile and blackcurrant.

### 2. Background Information

Advanced Glycation End Products (AGEs) are proteins that have been damaged by glycation; a non-enzymatic reaction between a sugar and a free amine group of the amino acids in proteins (Bailey A J, et al. “Mechanisms of maturation and ageing of collagen,” Mech Ageing Dev 106: 1-56 (1998); Bailey, A J. “Molecular mechanisms of ageing in connective tissues,” Mech Ageing Dev 122: 735-55 (2001)). This reaction begins with the formation of a sequence of early glycation products, Schiff bases and Amadori products that, over time, are chemically rearranged in a nearly irreversible process to form AGEs (Monnier V M, Mustata G T, Biernel K L, et al. “Cross-linking of the extracellular matrix by the maillard reaction in aging and diabetes: an update of a “a puzzle nearing resolution”,” Ann NY Acad Sci 1043: 533-44 (2005)).

Collagen, a protein that is critically important to maintain the strength, integrity and structure within the dermis, exhibits a slow turnover rate of about ten to fifteen years (Dyer D G, Dunn J A, Thorpe S R, et al. “Accumulation of Maillard reaction products in skin collagen in diabetes and aging,”J Clin Invest 91: 2463-2469 (1993); Jeanmaire C, Danous L, Pauly G. “Glycations during human dermal intrinsic and actinic ageing; an in vivo and in vitro model study,” Br J Dermatol 145: 10-18 (2001)). As a consequence of its long-lived structure, collagen is highly susceptible to the accumulation of AGES (Verzijl N, DeGroot J, Thorpe S R, et al. “Effects of collagen turnover on the accumulation of advanced glycation end products,”J Biol Chem 275: 39027-31(2000)).

AGEs accumulate in collagen as a function of chronological age (Jeanmaire, 2001; Hipkiss, Ark. “Accumulation of altered proteins and aging: causes and effects,” Exp Gerontol 41: 464-73 (2006)) and situations where blood glucose is elevated (Aronsen, D. “Crosslinking of glycated collagen in the pathogenesis of arterial and myocardial stiffening of aging and diabetes,”J Hypertension 21.1: 3-12 (2003)). In normal healthy individuals, the appearance of glycated collagen is first observed at the age of 20 and accumulates at a rate of about 3.7% yearly, reaching up to 50% by age 80 years (Jeanmaire, 2001; Dunn J A, McCance D R, Thorpe S R, et al. “Age dependent accumulation of N epsilon (carboxymethyl) lysine and N epsilon (carboxymethyl)hydroxylysine in human skin collagen,” Biochemistry 30: 1205-1210 (1991)). In individuals with frequently elevated blood glucose levels, AGE accumulation in skin becomes more rapid.

As such, compositions and methods for decreasing the blood sugar elevations, especially after sugar (sucrose) ingestion to decrease likelihood of a glycation reaction(s), and prevent accumulation of AGEs, are desired.

The features and advantages of the disclosure will be set forth in the description which follows, and in part will be apparent from the description, or may be learned by the practice of the disclosure without undue experimentation. The features and advantages of the disclosure may be realized and obtained by means of the combinations particularly pointed out in the appended claims.

### SUMMARY

The disclosure relates generally to dietary supplements comprising L-arabinose and one or more select compounds or extracts. The disclosure relates to a dietary supplement for reducing blood sugar and inhibiting glycation reaction(s) in the body and skin that result in Advanced Glycation End Products (AGEs). The composition of the disclosure combines a nutritionally effective amount of L-arabinose, and extracts of chamomile and blackcurrant.

Certain embodiments relate to a dietary supplement composition comprising: (i) an effective amount of L-arabinose; (ii) an effective amount of a chamomile extract; and (iii) an effective amount of a blackcurrant extract; wherein L-arabinose is present in an amount that is greater than about 50% by weight of the composition. The composition may be in a form of a liquid, tablet, pill, powder, fine granules, hard capsules, soft candy, jelly and other foods, or pharmaceuticals dosage forms. The composition may be in a form of a beverage. The composition may be administered at least once daily. The composition effectively reduces postprandial blood sugar levels in humans or animal subjects thereby decreasing the likelihood of a glycation reaction(s) in the skin of human or animal subjects.

Certain other embodiments relate to a dietary supplement composition comprising: (i) an amount of L-arabinose; (ii) an amount of a chamomile extract; and (iii) an amount of a blackcurrant extract; wherein the amounts of L-arabinose, the chamomile extract, and the blackcurrant extract are effective to produce at least one of: effective reduction in postprandial blood sugar levels in humans or animal subjects thereby decreasing the likelihood of a glycation reaction(s) in the human or animal subjects, and protect proteins from glycation reaction(s). In the dietary supplement composition, L-arabinose may be present in the composition in an amount that is greater than about 50% by weight. The composition may be in a form of a liquid, tablet, pill, powder, fine granules, hard capsules, soft candy, jelly and other foods, or pharmaceuticals dosage forms. The composition may be in a form of a beverage. The composition may be administered at least once daily. The composition effectively reduces postprandial blood sugar levels in humans or animal subjects thereby decreasing the likelihood of a glycation reaction(s) in the skin of human or animal subjects.

Certain further embodiments relate to a dietary supplement composition comprising: (i) an amount of L-arabinose; (ii) an amount of a chamomile extract; and (iii) an amount of a blackcurrant extract; wherein the amounts of L-arabinose, the chamomile extract, and the blackcurrant extract are effective to produce at least one of: effective reduction of skin glycation as measured by autofluorescence in human or animal subjects, and support a decrease in skin glycation. In the dietary supplement composition, L-arabinose may be present in the composition in an amount that is greater than about 50% by weight. The composition may be in a form of a liquid, tablet, pill, powder, fine granules, hard capsules, soft candy, jelly and other foods, or pharmaceuticals dosage forms. The composition may be in a form of a beverage. The composition may be administered at least once daily. The composition effectively reduces postprandial blood sugar levels in humans or animal subjects thereby decreasing the likelihood of a glycation reaction(s) in the skin of human or animal subjects.

Certain further embodiments relate to a method of managing blood sugar levels of a subject in need of thereof, comprising the step of administering the dietary supplement composition described herein to the subject, wherein the dietary supplement composition delivers the amounts of L-arabinose, the chamomile extract, and the blackcurrant extract to the subject. In the method, the composition may be administered in a form of a beverage. In the method, the composition may be administered at least once daily. The dietary supplement composition may comprise: (i) an effective amount of L-arabinose; (ii) an effective amount of a chamomile extract; and (iii) an effective amount of a blackcurrant extract; wherein L-arabinose is present in an amount that is greater than about 50% by weight of the composition.

Certain further embodiments relate to a method of decreasing glycation in skin of a subject in need of thereof, comprising administering the dietary supplement composition described herein to the subject, wherein the dietary supplement composition delivers the amounts of L-arabinose, the chamomile extract, and the blackcurrant extract to the subject. In the method, the composition may be administered in a form of a beverage. In the method, the composition may be administered at least once daily. The dietary supplement composition may comprise: (i) an effective amount of L-arabinose; (ii) an effective amount of a chamomile extract; and (iii) an effective amount of a blackcurrant extract; wherein L-arabinose is present in an amount that is greater than about 50% by weight of the composition.

Yet further embodiments relate to a method of preventing Advanced Glycation End Products (AGEs) in a skin of a human subject in need of thereof, comprising administering the dietary supplement composition described herein to the subject, wherein the dietary supplement composition delivers the amounts of L-arabinose, the chamomile extract, and the blackcurrant extract to the subject. In the method, the composition may be administered in a form of a beverage. In the method, the composition may be administered at least once daily. The dietary supplement composition may comprise: (i) an effective amount of L-arabinose; (ii) an effective amount of a chamomile extract; and (iii) an effective amount of a blackcurrant extract; wherein L-arabinose is present in an amount that is greater than about 50% by weight of the composition.

### BRIEF DESCRIPTION OF THE DRAWINGS

FIG. 1 depicts a graph change in blood glucose from baseline over 120 minutes after consuming (solid line) sucrose drink alone vs (dotted line) sucrose plus botanical mix.

FIG. 2 depicts a bar graph showing age reader measurements after 2 weeks of 3×/per day botanical mixed drink treatment.

FIG. 3 depicts the in vitro performance of two botanical combinations on glycation inhibition: (A) as compared to the botanicals alone; and (B) at various doses.

### DETAILED DESCRIPTION OF THE PRESENTLY PREFERRED EMBODIMENTS

It is to be understood that this invention is not limited to the particular compositions, methodology, or protocols described herein. Further, unless defined otherwise, all technical and scientific terms used herein have the same meaning as commonly understood to one of ordinary skill in the art to which this invention belongs. It is also to be understood that the terminology used herein is for the purpose of describing particular embodiments only, and is not intended to limit the scope of the present invention, which will be limited only by the claims.

In describing and claiming the subject matter of the disclosure, the following terminology will be used in accordance with the definitions set out below.

As used in this specification and the appended claims, the singular forms “a,” “an,” and “the” include plural referents unless the context clearly dictates otherwise. Thus, for example, reference to a dietary supplement comprising or containing “an excipient” includes a mixture of one or more of such excipients.

The terms “comprising,” “including,” “containing,” “characterized by,” and grammatical equivalents thereof are inclusive or open-ended terms that do not exclude additional, unrecited ingredients, elements or method steps.

The phrase “consisting of” and grammatical equivalents thereof exclude any element, step, or ingredient not specified in the claim.

The phrase “consisting essentially of” and grammatical equivalents thereof limit the scope of a claim to the specified ingredients, materials or steps and those that do not materially affect the basic and novel characteristic or characteristics of the claimed disclosure.

The terms “composition” or “formulation” refer to a product that treats, improves, promotes, increases, manages, controls, maintains, optimizes, modifies, reduces, inhibits, or prevents a particular condition associated with a natural state, biological process or disease or disorder. For example, a composition or a formulation described herein effectively reduces postprandial blood sugar levels in humans or animal subjects thereby decreasing the likelihood of a glycation reaction(s) in the human or animal subjects (i.e., inhibits glycation reaction(s)), protect proteins from glycation reaction(s), and decreases the rate by which AGEs are accumulated in the skin. The terms composition and formulation include, but are not limited to, pharmaceutical (i.e., drug), over-the counter (OTC), cosmetic, food, food ingredient or dietary supplement, drink compositions that include an effective amount of an extract, at least one component thereof, or a mixture thereof. Exemplary compositions and/or formulations include dietary supplements in a liquid form specifically prepared for human consumption, such as beverages (i.e., “drinks”). In certain embodiments, it might be possible to use the composition or formulation in a solid dose format or as an ingredient combination for a functional food rather than a beverage.

As used herein, the term “extract” or “botanical extract” refers to a solid, viscid, or liquid substance or preparation that includes an active ingredient(s) of a substance of plant, e.g., daisy-like plant of family Asteraceae (e.g., Matricaria chamomilla, Chamaemelum nobile); “chamomile extract”) and a woody shrub in the family Grossulariaceae, blackcurrant (e.g., Ribes nigrum, “blackcurrant extract”). The term “extract” is intended to include not only a crude extract produced from a plant, and by use of a solvent selected from among water, lower alcohols of 1 to 4 carbon atoms, such as methanol, ethanol, butanol, etc., ethylene, acetone, hexane, ether, chloroform, ethylacetate, butylacetate, dichloromethane, N,N-dimethylformamide (DMF), dimethylsulfoxide (DMSO), 1,3-butylene glycol, propylene glycol and a combination thereof, but also a fraction of the crude extract in such a solvent and/or a pure compound, composition, extract mixture, component of the extract, and/or active agent or ingredient, or a combination thereof. So long as it assures the extraction and preservation of the active ingredient(s), any extraction method may be employed.

As used herein, “effective amount” means an amount of an ingredient or component of the dietary supplement that is nontoxic, but sufficient to provide the desired effect and performance at a reasonable benefit/risk ratio attending any dietary supplement. An effective amount of L-arabinose, in combination with one or more select pure compounds, composition, extract mixture, component of the extract, and/or active agent or ingredient, or a combination thereof, is an amount sufficient to decrease blood sugar elevation after sugar ingestion (e.g., “postprandial” blood sugar, which means during or relating to the period after a meal, e.g., dinner or lunch) at a level sufficient to reduce the incidence of skin aging and/or other health issues caused by glycation reaction(s).

The phrase “to protect proteins from glycation reaction(s)” refers to the composition's or extract(s)'s ability to interfere, inhibit, reduce, and or prevent a non-enzymatic reaction between a sugar and a free amine group of the amino acids in proteins.

In an embodiment, a composition in accordance with the disclosure provides a nutritional supplement that operates to inhibit the glycation reaction(s). The dietary supplement is a composition that comprises L-arabinose and one or more pure compounds, composition, extract mixture, component of the extract, and/or active agent or ingredient, or a combination thereof of chamomile and blackcurrant. The combination of L-arabinose with the botanical extracts of chamomile and blackcurrant is designed to be delivered in a combination to inhibit glycation reactions and decrease the rate by which AGEs are accumulated in the skin.

The combination of L-arabinose, chamomile extract and blackcurrant extract surprisingly resulted in a decrease of the blood sugar elevations after sugar (sucrose) ingestion (i.e., postprandial blood sugar). Decreasing blood sugar elevations can help decrease the likelihood of a glycation reaction. Additionally, chamomile extract plus blackcurrant extract was shown in laboratory experiments to protect proteins from glycation reaction(s).

L-arabinose, also known as arabinose, pectin and sugar, an aldopentose, is a monosaccharide (molecular formula: C₅H₁₀O₅) molecule containing five carbon atoms that inhibits the breaking down of sucrose into fructose and glucose within the intestines of mammals. Arabinose is a naturally occurring sugar found in fruits and other plants, such as corn. L-arabinose is found in nature as a component of biopolymers, such as pectin and hemicellulose. L-arabinose can be harvested from a variety of sources including the stalks of corn and corn cobs. L-arabinose is a low-calorie sweetener, approved by the US Food and Drug Administration as an additive included in the health food. L-arabinose may be purchased from commercial sources (e.g., Cosun and Healtang).

Chamomile or camomile is the common name for several daisy-like plants of the family Asteraceae that are commonly used to make herb infusions to serve various medicinal purposes. Popular uses of chamomile preparations include treating hay fever, inflammation, muscle spasms, menstrual disorders, insomnia, ulcers, gastrointestinal disorders, and hemorrhoids. Major chemical compounds present within chamomile include apigenin and alpha-bisabolol. Other compounds in chamomile include: sesquiterpenes, terpenoids, flavonoids, coumarins such as herniarin and umbelliferone, phenylpropanoids such as chlorogenic acid and caffeic acid, flavones such as apigenin and luteolin, flavanols such as quercetin and rutin, and polyacetylenes. Apigenin has been previously shown to have chemopreventive effects against cancer cells in the laboratory (Patel, Deendayal; Shukla, Sanjeev; Gupta, Sanjay, “Apigenin and cancer chemoprevention: Progress, potential and promise (Review),” International Journal of Oncology 30 (1): 233-45 (2007), and alpha-bisabolol has been shown to have antiseptic properties, anti-inflammatory properties, and reduces pepsin secretion without altering secretion of stomach acid. In certain embodiments, the described compositions include a chamomile extract that includes, e.g., apegenin-7-glucoside and other flavonoids (standardized). The chamomile extract may be purchased from numerous commercial sources, such as Naturex, PLThomas. Also, Nutrilite grown chamomile may be used or chamomile extract prepared by know extraction method(s).

The blackcurrant (Ribes nigrum) is a woody shrub in the family Grossulariaceae grown for its piquant berries. It is native to temperate parts of central and northern Europe and northern Asia. The blackcurrant includes phytochemicals, such as polyphenols, in the fruit and seeds. Major anthocyanins in blackcurrant pomace are delphinidin-3-O-glucoside, delphinidin-3-O-rutinoside, cyanidin-3-O-glucoside, and cyanidin-3-O-rutinoside (Kapasakalidis, P G; Rastall, R A; Gordon, M H, “Extraction of polyphenols from processed black currant (Ribes nigrum L.) residues,” Journal of Agricultural and Food Chemistry 54 (11): 4016-21 (2006)), which are retained in the juice concentrate among other yet unidentified polyphenols (Mcdougall, G J; Gordon, S; Brennan, R; Stewart, D, “Anthocyanin-flavanol condensation products from black currant (Ribes nigrum L.),” Journal of Agricultural and Food Chemistry 53 (20): 7878-85 (2005); Nielsen, I L; Haren, G R; Magnussen, E L; Dragsted, L O; Rasmussen, S E, “Quantification of anthocyanins in commercial black currant juices by simple high-performance liquid chromatography. Investigation of their pH stability and antioxidative potency,” Journal of Agricultural and Food Chemistry 51 (20): 5861-6 (2003)). Also, blackcurrant seed oil is rich in nutrients, especially vitamin E and unsaturated fatty acids, including alpha-linolenic acid and gamma-linolenic acid (Traitler, H; Winter, H; Richli, U; Ingenbleek, Y, “Characterization of gamma-linolenic acid in Ribes seed,” Lipids 19 (12): 923-8 (1984)). In certain embodiments, the described compositions include a blackcurrant extract that includes, e.g., anthocyanins, apigenins (standardized), and total polyphenols. The natural vitamin E includes mixed tocopherols. The blackcurrant extract may be purchased from commercial sources, such as Artemis, Beijing Ginko Group, and Ningbo Green Health, or prepared by extraction.

In one embodiment, a composition may comprise a biologically effective composition comprising a range of about twenty parts to about one part of L-arabinose and one part of one or more of the select compounds or extracts, such as chamomile and blackcurrant extracts. In another embodiment, a composition may comprise a biologically effective composition comprising ten parts L-arabinose and one part of one or more of the select compounds or extracts, such as chamomile and blackcurrant extracts. In yet another embodiment, a composition may comprise a biologically effective composition comprising eight parts L-arabinose and one part of one or more of the select compounds or extracts, such as chamomile and blackcurrant extracts. In yet another embodiment, a composition may comprise a biologically effective composition comprising five parts L-arabinose and one part of one or more of the select compounds or extracts, such as chamomile and blackcurrant extracts. In another embodiment, a composition may comprise a biologically effective composition comprising three parts L-arabinose and one part of one or more of the select compounds or extracts, such as chamomile and blackcurrant extracts. In yet additional embodiment, a composition may comprise a biologically effective composition comprising one part L-arabinose and one part of one or more of the select compounds or extracts, such as chamomile and blackcurrant extracts. Further, L-arabinose may be present in the composition in an amount that is greater than about 50% by weight, or in an amount that may be between a range of about 55% to about 99% by weight, or in an amount that may be between a range of about 80% to about 95% by weight, or about 85% to about 90% by weight.

The select compounds or extracts of chamomile and blackcurrant may be present in the composition in an amount that is less than 50% by weight, or in an amount that may be within a range of about 49% to about 2% by weight, or in an amount that may be within a range of about 20% to about 5% by weight.

Certain embodiments relate to a composition that includes L-arabinose combined with chamomile extract and blackcurrant extract to produce a nutritional supplement that is capable of decreasing blood sugar elevations (e.g., postprandial blood sugar elevations) and can help decrease the likelihood of glycation reaction(s). Advantageously, compositions in accordance with the disclosure not only decrease blood sugar elevations but also provide other health benefits.

In certain embodiments, the effect of the ingredients of the composition may be at least additive, and may be synergistic.

For example, as noted previously, chamomile extract plus blackcurrant extract was shown in laboratory experiments to protect proteins from glycation reaction. This effect may be at least additive, or may be synergistic.

One potential advantage of the described compositions is improved digestive system function in the mammal. Because the L-arabinose inhibits the breakdown of sucrose in the system, the sucrose that is not broken down into fructose and glucose remains as a disaccharide throughout the digestive tract. The increased level of sucrose in the digestive tract provides an additional food source, i.e., a prebiotic, for use by probiotics and encourages increased numbers of probiotics. Probiotics are live microorganisms, including bacteria, which benefit the digestive tract. Examples of a probiotic include, but are not limited to, bifidobacterium, Clostridium butyricum, and the like. The increased food source (the unbroken down sucrose) leads to increased numbers of probiotics in the digestive system. The increased numbers of probiotics improve the health of the digestive system by reducing constipation and softening the stool to aid in proper function of the digestive system.

Advantageously, this shift to other non-sucrose macronutrients provides health benefits. Ingestion of compositions disclosed in the embodiments of the disclosure may result in reduced levels of glucose in the blood. Consequently, glycation reaction(s) in the body and skin may be inhibited. This in turn may be beneficial for preventing aging of the skin.

The dietary supplement of the disclosure can be formulated from using any commercially acceptable form of L-arabinose and extracts of chamomile and blackcurrant. The dietary supplement of the disclosure may include one or more binders, fillers, flavorings, buffers, gels, excipients, earners, or other compounds that facilitate the formulation or administration of the dietary supplement. The described composition can be formulated into tablets, granules, powders, gels, or liquids (a tablet as used herein refers to any form of a solid oral dosage, including but not limited to tablets, caplets, capsules, powders, and the like.).

Preferably, the composition is ingested into the body as a dietary supplement. As the composition passes through the intestines, the L-arabinose inhibits the breakdown of sucrose in the system and the other constituents of the composition provide additional health benefits, including those described above.

In certain embodiments, the composition is a liquid composition or a beverage. Examples of the types of beverages include but are not limited to soft drinks, carbonated beverages, nutritional drinks, nutritional shakes, juice drinks, lactic add drinks and other beverages.

In an embodiment, the dietary supplement may be administered in one, two or three dosages per day; however the supplement can be administered in other dosages and forms as desired.

The disclosure provides a dietary supplement composition by mixing appropriate unit doses of L-arabinose and extracts of chamomile and blackcurrant described herein to obtain a composition suitable for reducing blood sugar levels, e.g., postprandial blood sugar levels in humans or animal subjects in addition to other health benefits, such as decrease the likelihood of a glycation reaction.

Active new dietary supplement compositions of the disclosure may be produced by the procedures described herein or variations thereof, which will be apparent to one of skill in the art.

A further aspect of the disclosure is a pharmaceutical formulation comprising a composition as described above in a pharmaceutically acceptable carrier (e.g. an aqueous or a non-aqueous carrier).

A still further embodiment relates to a method of managing blood sugar levels (e.g., postprandial blood sugar levels) of a subject comprising the step of administering the described composition to a subject in need of thereof, wherein the composition delivers an effective amount of L-arabinose, chamomile extract, and blackcurrant extract to the subject.

Yet a further embodiment relates to a method of decreasing glycation in skin of a subject in need of thereof, comprising administering the described composition to the subject, wherein the composition delivers an effective amount of L-arabinose, chamomile extract, and blackcurrant extract to the subject.

Yes additional embodiment relates to a method of preventing AGEs in a skin of a human subject comprising administering the described composition to the subject, wherein the composition delivers an effective amount of L-arabinose, chamomile extract, and blackcurrant extract to prevent AGEs in the subject's skin.

A still further aspect of the disclosure is a method of reducing blood sugar levels (e.g., postprandial blood sugar levels) in a human or animal subject and/or of improving digestive health in the human or animal subject by administering a treatment effective amount (e.g. an amount effective to treat, reduce, or mitigate the breakdown of sucrose into glucose and fructose) of a composition as described above.

In the above methods, the composition may be administered in a form of a beverage. In the above methods, the composition may be administered at least once daily.

The following are illustrative examples of formulations and compositions according to this invention. Although the examples use only selected compounds and formulations, it should be understood that the following examples are illustrative and not limited.

### EXAMPLES

The practices and procedures adopted during the conduct of this study were consistent with the International Conference of Harmonization (ICH) and Good Clinical Practices (GCP). Each subject was provided a copy of the Informed Consent form (ICF) and prior to testing, each subject gave voluntary written consent to participate by signing the ICF.

### Example 1: Blood Glucose Measurement Methods and Results

A 3 week repeated measure controlled study was conducted in which each subject served as their own control. Subjects fasted 12 hours prior to the first day of experimentation, after being encouraged to drink water as needed. On the day of the test, baseline glucose values were measured via an Advanced Glucose Meter. The fasted subjects then ingested 75 g sucrose dissolved in 300 mL warm water over a 5 minute period. Blood glucose values were then measured at 15, 30, 45, 60, 90, and 120 minutes following the ingestion of the sucrose-water drink mix.

Subjects were given a two-week wash out period between experimental days. On day two of experimentation, again the subjects arrived to the testing location following a 12 hour fast, only water allowed to drink. The same procedure was followed as experimentation day 1 with baseline glucose values measured by the same Advanced Glucose Meter utilized previously. Subjects then consumed a mixture of 75 g sucrose combined with a L-arabinose/chamomile/black currant extract blend, formulated as shown in Table 1 below, dissolved in 300 mL warm water over a five minute period.

TABLE 1

| Ingredient Description | Function | MG |
|---|---|---|
| Chamomile Extract | Active | 100.00 |
| Black Current | Active | 15.00 |
| Vitamin E | Active | 8.00 |
| Inulin | Mouthfeel | 899.35 |
| Reb A (Stevia) | Sweetener | 15.00 |
| Arabinose | Active | 1050.00 |
| Malic Acid | Acid ulant | 185.00 |
| MCT Oil | Processing Aid | 6.35 |
| Natural Flavor | Flavor | 392.00 |
| Natural Color | Color | 87.00 |
| Total Weight | | 2757.70 |

The Advanced Glucose Meter was utilized to measure blood glucose values 15, 30, 45, 60, 90, and 120 minutes following the ingestion of 75 gm sucrose+L-arabinose/chamomile/black current extract blend drink mix.

FIG. 1 depicts a change in blood glucose from baseline over 120 minutes after consuming (circles, solid line) sucrose drink alone vs (squares, dotted line) sucrose plus botanical mix. Sucrose drink with botanical mix shows blunted post-prandial glucose response in comparison to sucrose alone. Glucose measures at times 15, 30 and 45 minutes after drink were significantly lower when subjects consumed sucrose with botanical mix compared to that seen when consuming sucrose only (p<0.05, two-tailed paired t-test). Calculating area under the curve (AUC) of the total 120 minute mean change in blood glucose demonstrated a significant reduction of total post-prandial glucose response following sucrose with botanical mix than the mean blood glucose seen after sucrose alone with p=0.01

### Example 2: Age Reader Measurement Methods and Results

The accumulation of Advanced Glycation End Products (AGEs) advances over time normally as one ages, but can also be accelerated in situations of oxidative stress or poorly controlled glycemia. As such, AGE accumulation is thought to play a role in the pathogenesis of chronic, age-related diseases (Schleicher E, Wagner E, Nerlich A G. “Increased accumulation of the glycoxidation product N-carboxymethyllysine on human tissues in diabetes and aging,” J Clin Invest 99:457-468 (1997)). Several predominate AGE accumulate in the skin and are fluorescent at specific wavelengths and can be measured using an autofluorescent Reader such as AGE Reader. With this non-invasive measuring tool, autofluorescence is defined as the average fluorescence per nm over the entire emission spectrum (420-600 nm) as a ratio of the average fluorescence per nm over the 300-420 nm range. These measures have been highly correlated with skin biopsies analyzed for collagen-link fluorescence and specific AGE (Meerwaldt R, Graaff R, Oomen P H N, “Simple non-invasive assessment of advanced glycation end product accumulation,” Diabetologia 47:1324-1330 (2004)).

In this study the widely accepted AGE Reader device was used as a non-invasive measuring tool to assess the effects of consuming a combination botanical drink designed to inhibit glycation reactions in-vivo. All measurements were conducted at room temperature on the exposed volar side of the arm. Autofluorescence (AFR) was measured three times at each site. Subjects were provided unlabeled samples with instructions to consume the botanical mix (Table 1 above) with water three times a day for 14 days. At the conclusion of the 2 week experimental period, the subjects returned to the study site to repeat the AGE Reader measurements at the same spot on their volar side of the forearm. The AFR measurements were tallied, averaged, and compared between the baseline time point and those obtained after two weeks of 3 times a day botanical extract mix, with each subject serving as their own control in a repeated measure design. The average baseline AFR was 1.971 (SEM±0.19) while the average AFR after consuming botanical mix 3 time a day for two weeks was reduced to 1.540 (SEM±0.04). A paired two tail t-test revealed this reduction was statistically significant with a p value<0.05 (p=0.025).

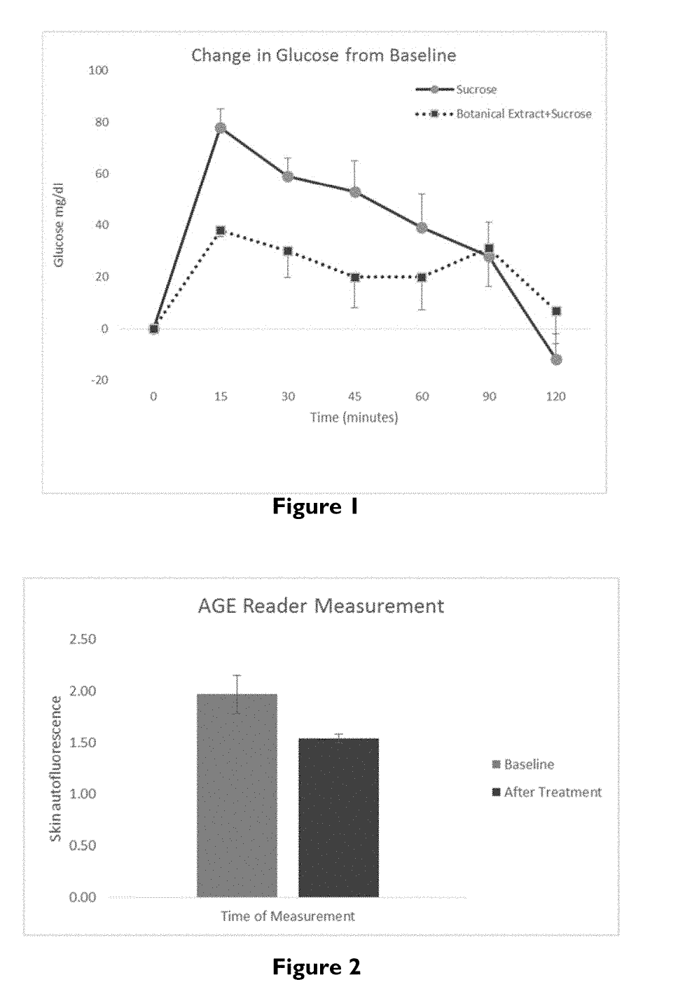

FIG. 2 depicts the age reader measurements after 2 weeks of 3×/per day botanical mixed drink treatment. The average baseline was 1.971 (SEM±0.19) while the average after consuming botanical mix 3 time a day for two weeks was reduced to 1.540 (SEM±0.04). A paired two tail t-test revealed this reduction was statistically significant with a p value<0.05 (p=0.025).

### Example 3: Glycation Inhibition (In Vitro) Methods and Results

Glycation inhibition at the molecular level can help prevent sugars from binding proteins and reduce the progress of the glycation reaction from early glycation products (Schiff base) to later, more intermediate glycation products (Amadori product). The most well-known glycation inhibitor is aminoguanadine but unfortunately, this product demonstrated adverse effects in clinical trials and thus is not a viable therapy in humans. However, as the gold standard for antiglycation activity, aminoguanadine serves as the positive control to measure antiglycation activity of other biological substances.

Here, the extracts—Chamomile and Black Current, or Chamomile, Black Current and Arabinose—were solubilized in DMSO and diluted to final test concentrations with PBS. Diluted extract combinations were combined with bovine serum albumin and ribose and incubated at 65° C. for 48 hours. Following the incubation, the plates were centrifuged at 200×g for two minutes followed by fluorescent measurements with the Spectramax M5 at 340 nm excitation and 410 emission. Results were normalized to the glycation inhibitor aminoguanadine, with all botanical combinations tested at least in duplicate. Results indicate both combinations of extracts tested inhibit glycation at nearly identical levels regardless of whether or not the arabinose was included in the formula, suggesting the entire mix is responsible for the effect. The combinations of extracts demonstrated a dose response curve with a maximum of approximately 60% glycation inhibition at the top dose combination in comparison to the aminoguanadine control.

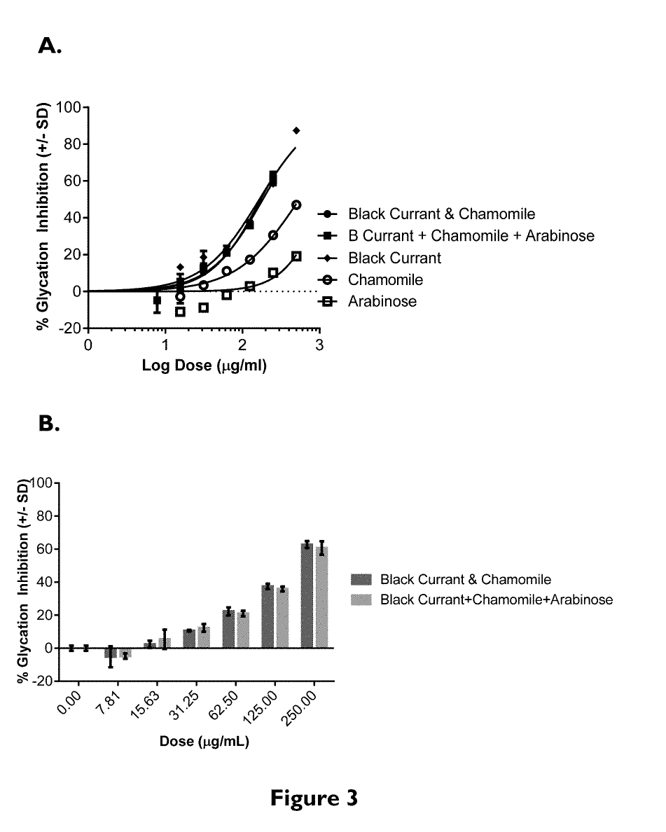

FIGS. 3A-B depict in vitro performance of two botanical combinations on glycation inhibition. Extracts were solubilized in DMSO and diluted to final combination with PBS. Dilute extract mix was combined with BVA and incubated for 48 hours, after which the response was normalized to the response of aminoguanadine. The combination of black current and chamomile (circles (FIG. 3A), dark bars (FIG. 3B)) did not differ in effect from the combination of black current, chamomile and arabinose (squares (FIG. 3A), light bars (FIG. 3B)). Both combinations achieve a glycation inhibition percentage of just over 60% at the top dose.

It is therefore intended that the foregoing detailed description be regarded as illustrative rather than limiting, and that it be understood that it is the following claims, including all equivalents, that are intended to define the spirit and scope of this invention.

1. A dietary supplement composition comprising:
(i) an effective amount of L-arabinose;
(ii) an effective amount of a chamomile extract; and
(iii) an effective amount of a blackcurrant extract;
wherein L-arabinose is present in an amount that is greater than about 50% by weight of the composition.

2. The dietary supplement composition of claim 1, wherein the composition is in a form of a liquid, tablet, pill, powder, fine granules, hard capsules, soft candy, jelly and other foods, or pharmaceuticals dosage forms.

3. The dietary supplement composition of claim 1, wherein the composition is in a form of a beverage.

4. The dietary supplement composition of claim 1, wherein the composition is administered at least once daily.

5. The dietary supplement composition of claim 1, wherein the composition effectively reduces blood sugar levels in humans or animal subjects thereby decreasing the likelihood of a glycation reaction(s) in the skin of human or animal subjects.

6. A method of managing blood sugar levels of a subject in need of thereof, comprising the step of administering the composition of claim 1 to the subject, wherein the composition delivers the effective amount of L-arabinose, the effective amount of the chamomile extract, and the effective amount of the blackcurrant extract to the subject.

7. The method of claim 6, wherein the composition is administered in a form of a beverage.

8. The method of claim 6, wherein the composition is administered at least once daily.

9. A method of decreasing glycation in skin of a subject in need of thereof, comprising administering the composition of claim 1 to the subject, wherein the composition delivers the effective amount of L-arabinose, the effective amount of the chamomile extract, and the effective amount of the blackcurrant extract to the subject.

10. The method of claim 9, wherein the composition is administered in a form of a beverage.

11. The method of claim 9, wherein the composition is administered at least once daily.

12. A method of preventing Advanced Glycation End Products (AGEs) in a skin of a human subject in need of thereof, comprising administering the composition of claim 1 to the subject, wherein the composition delivers the effective amount of L-arabinose, the effective amount of the chamomile extract, and the effective amount of the blackcurrant extract to the subject.

13. The method of claim 12, wherein the composition is administered in a form of a beverage.

14. The method of claim 12, wherein the composition is administered at least once daily.

15. A dietary supplement composition comprising:
(i) an amount of L-arabinose;
(ii) an amount of a chamomile extract; and
(iii) an amount of a blackcurrant extract;
wherein the amounts of L-arabinose, the chamomile extract, and the blackcurrant extract are effective to produce at least one of: effective reduction in postprandial blood sugar levels in humans or animal subjects thereby decreasing the likelihood of a glycation reaction(s) in the human or animal subjects, and protect proteins from glycation reaction(s).

16. The dietary supplement composition of claim 15, wherein L-arabinose is present in the composition in an amount that is greater than about 50% by weight.

17. A dietary supplement composition comprising:
(i) an amount of L-arabinose;
(ii) an amount of a chamomile extract; and
(iii) an amount of a blackcurrant extract;
wherein the amounts of L-arabinose, the chamomile extract, and the blackcurrant extract are effective to produce at least one of: effectively reducing skin glycation as measured by autofluorescence in human or animal subjects, and support a decrease in skin glycation.

18. The dietary supplement composition of claim 17, wherein L-arabinose is present in the composition in an amount that is greater than about 50% by weight.
